(19) United States
(12) Patent Application Publication
(10) Pub. No.: US 2020/0260936 A1
(43) Pub. Date: Aug. 20, 2020

(54) LIGHT SOURCE APPARATUS FOR ENDOSCOPE

(71) Applicant: OLYMPUS CORPORATION, Tokyo (JP)

(72) Inventors: So MATSUKA, Tokyo (JP); Minoru SATO, Tokyo (JP)

(73) Assignee: OLYMPUS CORPORATION, Tokyo (JP)

(21) Appl. No.: 16/865,453

(22) Filed: May 4, 2020

Related U.S. Application Data
(63) Continuation of application No. PCT/JP2018/026591, filed on Jul. 13, 2018.

(30) Foreign Application Priority Data
Nov. 7, 2017 (JP) 2017-214808

(51) Int. Cl. A61B 1/00; A61B 1/06

(52) U.S. Cl. CPC A61B 1/00126; A61B 1/07; A61B 1/0669; A61B 1/00006

(57) ABSTRACT

A light source apparatus for endoscope includes a light source configured to generate illumination light, a light source connector receiving member into or from which a light source connector provided in a connector of an endoscope is inserted or removed, a light blocking unit in which a light blocking plate configured to close an opening portion of the light source connector receiving section is disposed to be openable and closable, and an assist unit including a movable member configured to interlock with the insertion or removal of the light source connector into or from the light source connector receiving section by abutting on the light blocking plate and close the opening portion of the light source connector receiving section to press the light blocking plate in a closing direction in which the illumination light from the light source is blocked.

### CROSS REFERENCE TO RELATED APPLICATION

This application is a continuation application of PCT/JP2018/026591 filed on Jul. 13, 2018 and claims benefit of Japanese Application No. 2017-214808 filed in Japan on Nov. 7, 2017, the entire contents of which are incorporated herein by this reference.

### BACKGROUND OF INVENTION

### 1. Field of the Invention

The present invention relates to a light source apparatus for endoscope to which an endoscope connector of an endoscope is detachably attached and contains an illumination light source.

### 2. Description of the Related Art

Conventionally, in a medical field, an endoscope capable of observing organs within a body cavity, for example, by inserting an elongated insertion section into the body cavity has been widely used.

The endoscope has a function of irradiating illumination light into the body cavity that external light does not enter. As the illumination light, light from a light source provided in the light source apparatus for endoscope as an external apparatus is introduced into the endoscope.

The endoscope includes an endoscope connector detachably attached to a receptacle section in the light source apparatus for endoscope, and illumination light is introduced into the endoscope via the endoscope connector.

The conventional light source apparatus for endoscope is disclosed in Japanese Patent Application Laid-Open Publication No. 2007-20855, for example, and a technique for preventing illumination light from leaking even if a light source connector (light carrying bundle sleeve) provided in a connector of an endoscope is removed from a light source connection hole while a light source is lighting up has been known.

### SUMMARY OF THE INVENTION

A light source apparatus for endoscope according to an aspect of the present invention includes a light source configured to generate illumination light, a light source connector receiving member into or from which a light source connector provided in a connector of an endoscope is inserted or removed, a light blocking plate configured to be movable between a position where an opening portion of the light source connector receiving member is closed and a position where the opening portion is exposed and including an abutment surface configured to abut on the light source connector, and a movable member configured to abut on a rear surface of the abutment surface of the light blocking plate, press the light blocking plate at the position where the opening portion is closed from a side of the rear surface in a closing direction in which the opening portion is closed with the light source connector not inserted into the light source connector receiving member, and make the light blocking plate movable to the position where the opening portion is exposed against the pressing in the closing direction when the light source connector inserted into the light source connector receiving member presses the light blocking plate by abutting on the abutment surface of the light blocking plate.

### BRIEF DESCRIPTION OF THE DRAWINGS

FIG. 1 is a perspective view illustrating a configuration of an endoscope and a light source apparatus for endoscope;

FIG. 2 is a cross-sectional view of a connector section provided with a light blocking unit;

FIG. 3 is a cross-sectional view of a connector section to which a connector of the endoscope remains connected;

FIG. 4 is a perspective view illustrating a configuration of the light blocking unit;

FIG. 5 is a perspective view illustrating a configuration of a light blocking plate unit;

FIG. 6 is a cross-sectional view illustrating the configuration of the light blocking plate unit;

FIG. 7 is an exploded perspective view illustrating a configuration of an assist unit;

FIG. 8 is a perspective view illustrating the configuration of the assist unit;

FIG. 9 is a side view illustrating a configuration of a turning member;

FIG. 10 is a cross-sectional view in an initial state where a light source connector of the connector of the endoscope is inserted into a light source connector receiving section;

FIG. 11 is a cross-sectional view in a process state where the light source connector of the connector of the endoscope is inserted into the light source connector receiving section;

FIG. 12 is a cross-sectional view in an in-use state where the light source connector of the connector of the endoscope is inserted into the light source connector receiving section;

FIG. 13 is a side view illustrating a configuration of a turning member in a first modification;

FIG. 14 is a cross-sectional view in an initial state where a light source connector of a connector of an endoscope is inserted into a light source connector receiving section in the first modification;

FIG. 15 is a cross-sectional view in an in-use state where the light source connector of the connector of the endoscope is inserted into the light source connector receiving section in the first modification;

FIG. 16 is an exploded perspective view illustrating a configuration of an assist unit in a second modification;

FIG. 17 is a cross-sectional view in an initial state where a light source connector of a connector of an endoscope is inserted into a light source connector receiving section in a third modification;

FIG. 18 is a cross-sectional view in an in-use state where the light source connector of the connector of the endoscope is inserted into the light source connector receiving section in the third modification;

FIG. 19 is a cross-sectional view in an initial state where a light source connector of a connector of an endoscope is inserted into a light source connector receiving section in a fourth modification; and

FIG. 20 is a cross-sectional view in an in-use state where the light source connector of the connector of the endoscope is inserted into the light source connector receiving section in the fourth modification.

### DETAILED DESCRIPTION OF THE PREFERRED EMBODIMENT

A preferred embodiment of the present invention will be described below with reference to the drawings.

In the drawings used for the following description, a scale is made to differ for each of components to make the size of the component recognizable on the drawings, and the present invention is not limited only to a number of the components, respective shapes of the components, a ratio of the respective sizes of the components, and a relative positional relationship of the components, described in the drawings. In the following description, a vertical direction viewed toward a plane of the drawing may be described as an upper part and a lower part of the component.

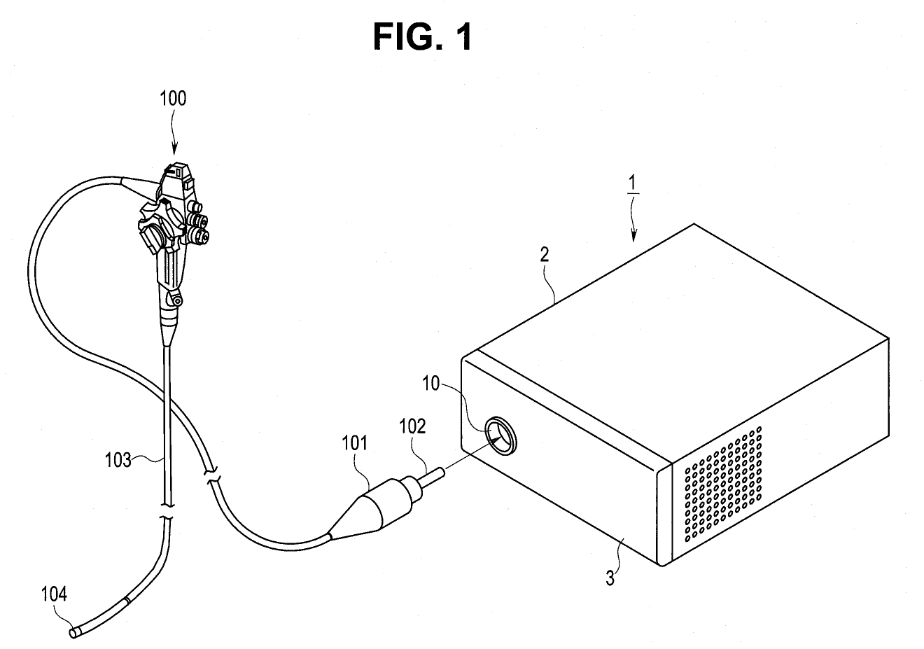

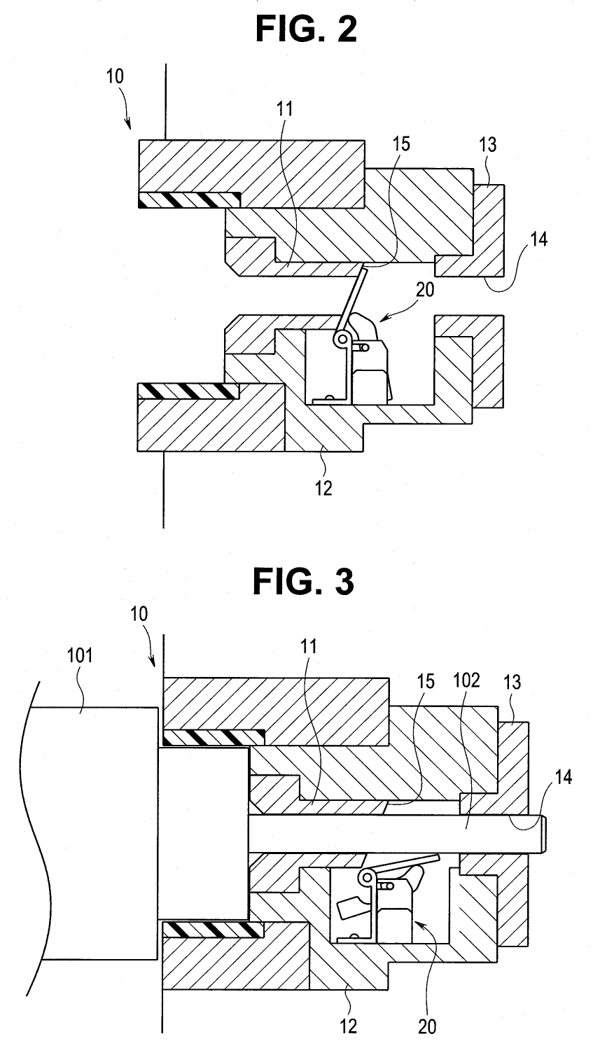

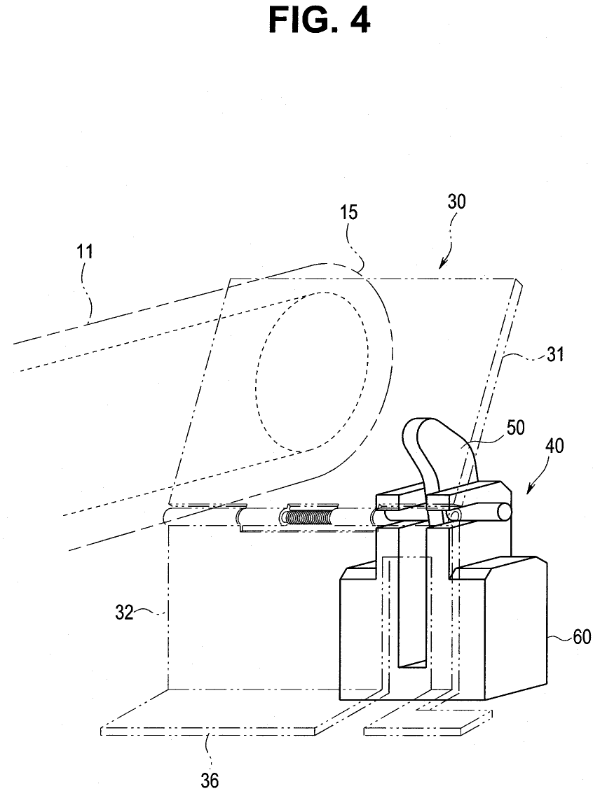

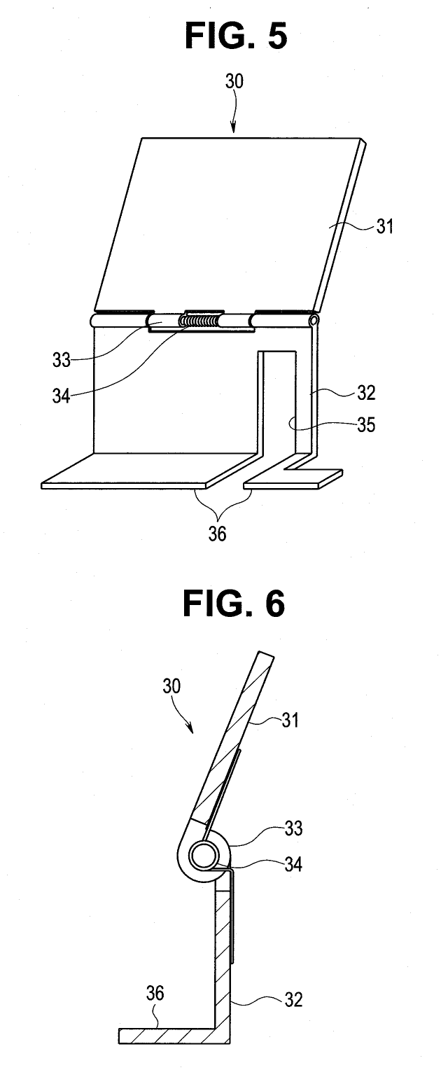

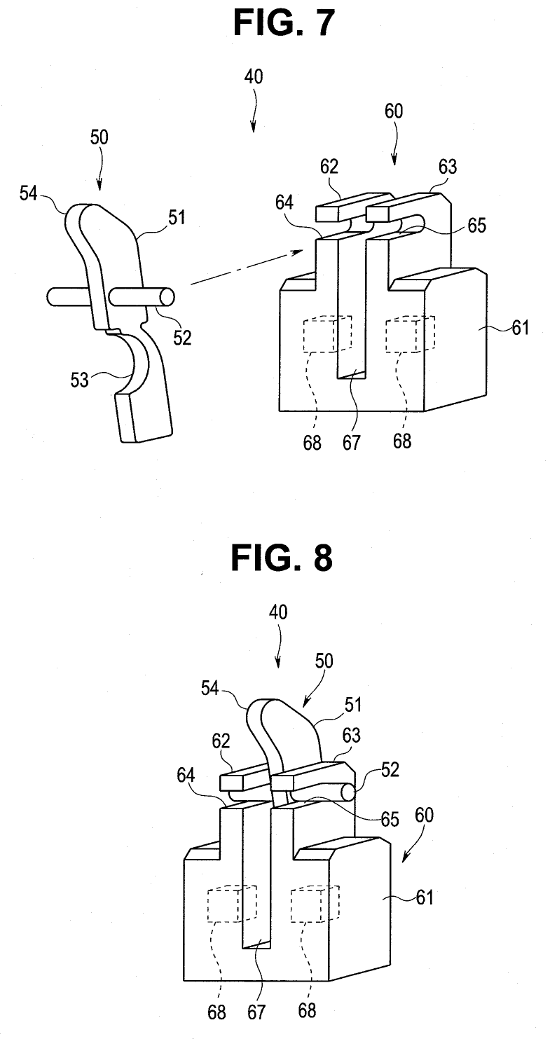

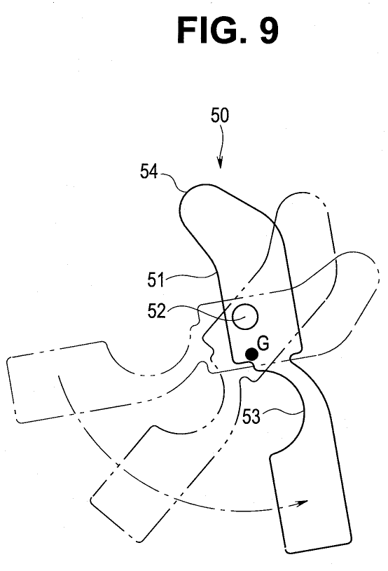

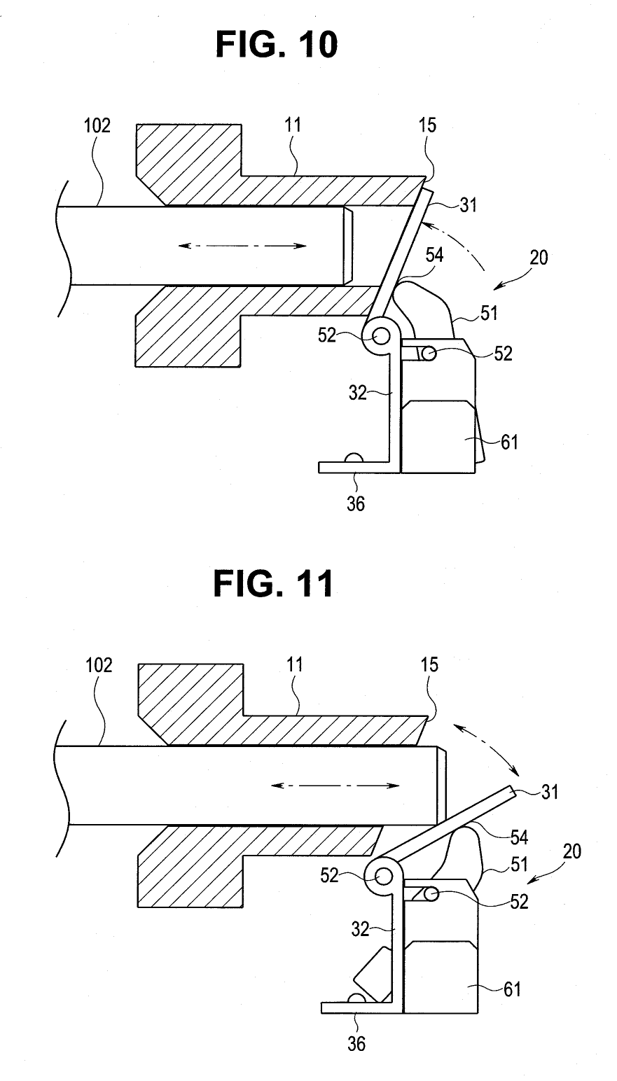

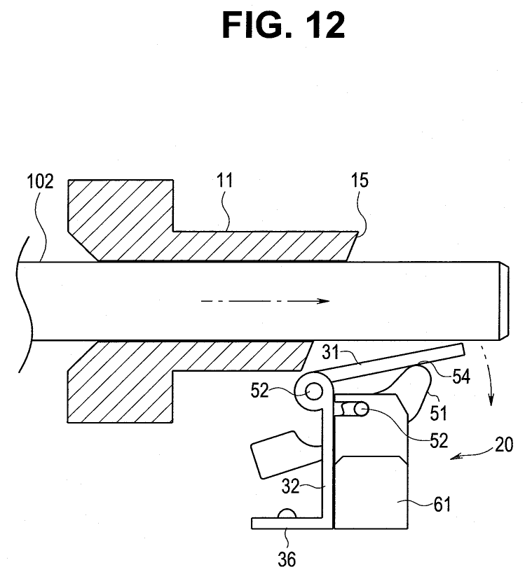

FIG. 1 is a perspective view illustrating a configuration of an endoscope and a light source apparatus for endoscope, FIG. 2 is a cross-sectional view of a connector section provided with a light blocking unit, FIG. 3 is a cross-sectional view of a connector section to which a connector of the endoscope remains connected, FIG. 4 is a perspective view illustrating a configuration of the light blocking unit, FIG. 5 is a perspective view illustrating a configuration of a light blocking plate unit, FIG. 6 is a cross-sectional view illustrating the configuration of the light blocking plate unit, FIG. 7 is an exploded perspective view illustrating a configuration of an assist unit, FIG. 8 is a perspective view illustrating the configuration of the assist unit, FIG. 9 is a side view illustrating a configuration of a turning member, FIG. 10 is a cross-sectional view in an initial state where a light source connector of the connector of the endoscope is inserted into a light source connector receiving section, FIG. 11 is a cross-sectional view in a process state where the light source connector of the connector of the endoscope is inserted into the light source connector receiving section, and FIG. 12 is a cross-sectional view in an in-use state where the light source connector of the connector of the endoscope is inserted into the light source connector receiving section.

A light source apparatus for endoscope 1 as a medical electronic apparatus according to the present embodiment illustrated in FIGS. 1 and 2 is an apparatus used together with an endoscope 100. The light source apparatus for endoscope 1 includes an image processing apparatus configured to be communicable by wire or wirelessly with an image pickup apparatus included in the endoscope 100, generate an observation image based on a signal to be inputted from the image pickup apparatus, and output the generated observation image to an image display apparatus not illustrated.

The image pickup apparatus in the endoscope 100 has a configuration in which an optical image of a subject is picked up. A configuration of the endoscope 100 is known, and hence detailed description is omitted.

In the present embodiment, the light source apparatus for endoscope 1 includes a receptacle-shaped connector section 10 in a front panel 3 in a housing 2 as an example. A plug-shaped connector 101 of the endoscope 100 is connected to the connector section 10. Note that an elongated light source connector 102 having a substantially pillar shape is provided to extend in the connector 101.

The light source apparatus for endoscope 1 is electrically connected to an image pickup apparatus including an image pickup device such as a CCD or a CMOS provided in the endoscope 100 via the connector section 10, to control an operation of and supply power to the image pickup apparatus.

The light source apparatus for endoscope 1 includes a light source not illustrated configured to generate illumination light for irradiating an object of the image pickup apparatus. Examples of the light source include a halogen lamp, a xenon lamp, and an LED, and illumination light is incident on a light guide fiber bundle provided in the endoscope 100 via the connector 101 of the endoscope 100 connected to the connector section 10.

The light guide fiber bundle has its one end disposed within the light source connector 102 and has its other end disposed within a distal end portion 104 of an insertion section 103 of the endoscope 100. Illumination light is irradiated onto the subject from an illumination window provided in the distal end portion 104.

Note that the housing 2 in the light source apparatus for endoscope 1 is in a shape of a box having a rectangular parallelepiped shape, and the connector section 10 is arranged in the front panel 3 standing substantially upright with respect to a ground surface with the housing 2 placed on a surface substantially parallel to the ground surface in a usable posture.

The connector section 10 in the light source apparatus for endoscope 1 will be described below.

As illustrated in FIGS. 2 and 3, in the connector section 10, a substantially cylindrical light source connector receiving section 11 into or from which the light source connector 102 provided in the connector 101 of the endoscope 100 is inserted or removed is fitted in a block-shaped frame 12. The frame 12 is provided with a heat sink 13 having a hole section 14, via which an end portion of the light source connector 102 is inserted into the apparatus, formed therein, for example.

In the light source connector receiving section 11, an end surface 15 within the apparatus is an inclined surface inclined at a predetermined angle to a longitudinal axis. Note that the end surface 15 may be a surface in a vertical direction perpendicular to a longitudinal direction of the light source connector receiving section 11.

In the frame 12, a light blocking unit 20 configured to block light from a light source by closing an opening portion on the end surface 15 of the light source connector receiving section 11 is disposed with the connector 101 of the endoscope 100 not inserted into the connector section 10.

The light blocking unit 20 is movable by abutting on the light source connector 102 to be inserted into the light source connector receiving section 11, and the light source connector 102 is inserted into the light source apparatus for endoscope 1.

A configuration of the light blocking unit 20 in the present embodiment will be described in detail below.

As illustrated in FIG. 4, the light blocking unit 20 includes a light blocking plate unit 30 including an openable and closable light blocking plate 31 configured to close the opening portion on the end surface 15 of the light source connector receiving section 11 and an assist unit 40 configured to press the light blocking plate 31 in the light blocking plate unit 30 in a closing direction toward the end surface 15 of the light source connector receiving section 11 under its own weight.

The light blocking plate unit 30 includes a light blocking plate 31 having a substantially rectangular shape and a support plate 32 having an L shape in cross section, as illustrated in FIGS. 5 and 6. The light blocking plate 31 and the support plate 32 are turnably connected to each other by a hinge section 33.

A torsion spring 34 as an urging member is interposed in the hinge section 33, and the light blocking plate 31 is urged to be upright vertically upward with respect to the support plate 32 by the torsion spring 34.

The support plate 32 has a notched groove section 35 formed in its one side portion. The groove section 35 is for avoiding the assist unit 40 contacting the support plate 32. A bottom section 36 folded in an L shape is pierced with a plurality of screw holes not illustrated, and is to be a bottom surface portion to be secured to the frame 12 with screws.

The assist unit 40 includes a turning member 50 as a movable member and a support base 60 configured to turnably support the turning member 50, as illustrated in FIGS. 7 and 8.

In the turning member 50, a main body section 51 as a plate-shaped block body is provided with a turning shaft 52. The main body section 51 has an abutment end portion 54 in its upper end portion, and a circular arc-shaped recess 53 is formed below the turning shaft 52.

The abutment end portion 54 is a site configured to abut on the light blocking plate 31. The recess 53 is formed to avoid contact with the support plate 32 in the light blocking plate unit 30 when the main body section 51 has been turned around the turning shaft 52. Note that a lower portion of the main body section 51 enters the groove section 35 formed in the support plate 32 in the light blocking plate unit 30 to avoid contact with the light blocking plate unit 30.

The support base 60 includes two arm sections 62 and 63 provided to extend with a predetermined separation distance therebetween on an upper side of a base main body section 61, and turning holding grooves 64 and 65 on which the turning shaft 52 in the turning member 50 is mounted are respectively formed in the two arm sections 62 and 63.

Each of the turning holding grooves 64 and 65 has a groove width slightly smaller than a diameter of the turning shaft 52, and has a circular arc surface of substantially the same size as the size of an outer periphery of the turning shaft 52 formed in its groove bottom portion. In other words, when the turning shaft 52 is pressed into the groove bottom portion of each of the turning holding grooves 64 and 65, the turning member 50 is turnably held without dropping off the support base 60.

In the support base 60, a groove section 67 configured to house a lower portion of the turning member 50 is formed along respective opposing surfaces of the two arm sections 62 and 63, and photosensors 68 as detection sections are contained with the groove section 67 sandwiched therebetween.

The photosensor 68 is electrically connected to a control section not illustrated provided in the light source apparatus for endoscope 1. The photosensor 68 detects the main body section 51 in the turning member 50 configured to be turned within the groove section 67 in the support base 60 and outputs a detection result to the control section.

Note that the turning member 50 has a center of gravity G set vertically below the turning shaft 52, as illustrated in FIG. 9, and takes a posture in which the abutment end portion 54 in the main body section 51 is positioned on an upper side under its own weight while remaining assembled to the support base 60.

The assist unit 40 in which the turning member 50 is assembled to the support base 60 is fixed to the frame 12 with screws, for example, such that the turning member 50 in the assist unit 40 abuts on a region outside a region where the light blocking plate 31 in the light blocking plate unit 30 abuts on the end surface 15 of the light source connector receiving section 11, as illustrated in FIG. 4.

An operation of the light blocking unit 20 performed when the connector 101 of the endoscope 100 is inserted into or removed from the connector section 10 in the light source apparatus for endoscope 1 configured as described above will be described below.

As illustrated in FIG. 10, in an initial state where the light source connector 102 of the connector 101 of the endoscope 100 does not contact the light blocking plate 31 in the light blocking plate unit 30 at the time of insertion or removal when the light source connector 102 is inserted into or removed from the light source connector receiving section 11 (including the time of not-in-use), a stress to be turned in a direction in which the abutment end portion 54 in the main body section 51 abuts on and pushes up the light blocking plate 31 under an own weight of the turning member 50 is generated in addition to an urging force of the torsion spring 34.

As a result, the light blocking plate 31 is pressed in a closing direction toward the end surface 15 of the light source connector receiving section 11, and the opening portion of the light source connector receiving section 11 is closed. As a result, there occurs a state where illumination light at the time of lighting of the light source is reliably blocked without leaking out of the light source connector receiving section 11.

As illustrated in FIG. 11, in a process state where the light source connector 102 is inserted into or removed from the light source connector receiving section 11, there occurs a state where the light source connector 102 contacts the light blocking plate 31 so that the light blocking plate 31 is turned away from the end surface 15 of the light source connector receiving section 11 against the urging force of the torsion spring 34 and the stress to be turned under the own weight of the turning member 50.

Note that in an in-use state where the light source connector 102 is inserted into the light source connector receiving section 11 so that the connector 101 of the endoscope 100 is connected to the connector section 10 in the light source apparatus for endoscope 1, as illustrated in FIG. 12, there occurs a state where the light blocking plate 31 is urged and turned to fall downward against the urging force of the torsion spring 34 and the stress to be turned under the own weight of the turning member 50.

At this time, when the photosensor 68 provided in the support base 60 detects an open state where a lower end portion of the main body section 51 is out of the groove section 67 in the support base 60 by the turning of the turning member 50, it is detected that the connector 101 of the endoscope 100 has been connected to the connector section 10.

Note that when the lower end portion of the main body section 51 is within the groove section 67 in the support base 60, a state where the connector 101 of the endoscope 100 is disconnected to the connector section 10 is detected as a close state by the photosensor 68.

The light source apparatus for endoscope 1 may be configured to control ON/OFF of lighting of the light source depending on a detection result by the photosensor 68 of a state where the connector 101 of the endoscope 100 is connected/disconnected to the connector section 10.

As described above, the light source apparatus for endoscope 1 according to the present embodiment is configured such that illumination light reliably does not leak because the light blocking plate 31 in the light blocking plate unit 30 blocks the illumination light by abutting on the end surface 15 of the light source connector receiving section 11 upon receiving the urging force of the torsion spring 34 and the stress to be turned under the own weight of the turning member 50 even if the light source is lighting up in an initial state at the time of not-in-use and at the time of insertion or removal of the connector 101 of the endoscope 100.

Even if the urging force is weakened or lost because a malfunction or the like occurs in the torsion spring 34, the light blocking plate 31 is turned to be pushed up under the own weight of the turning member 50 by the assist unit 40 so that the opening portion of the light source connector receiving section 11 is closed.

On the other hand, even if the stress for pushing up the light blocking plate 31 is weakened or lost because a malfunction or the like occurs in the assist unit 40, the light blocking plate 31 is turned to be pushed up by the urging force of the torsion spring 34 in the light blocking plate unit 30 so that the opening portion of the light source connector receiving section 11 is closed.

Accordingly, the light source apparatus for endoscope 1 is configured such that illumination light from the light source at the time of lighting can be more reliably blocked not to leak when the connector 101 of the endoscope 100 is inserted or removed, including the time of not-in-use, by the light blocking unit 20 configured to urge the light blocking plate 31 and close the opening portion of the light source connector receiving section 11.

Note that the light blocking plate unit 30 need not necessarily be provided with the torsion spring 34 configured to urge the light blocking plate 31 in a direction in which the opening portion of the light source connector receiving section 11 is closed. In other words, the light blocking plate 31 may be configured to receive a stress to be turned in the direction in which the opening portion of the light source connector receiving section 11 is closed only by the assist unit 40.

### (First Modification)

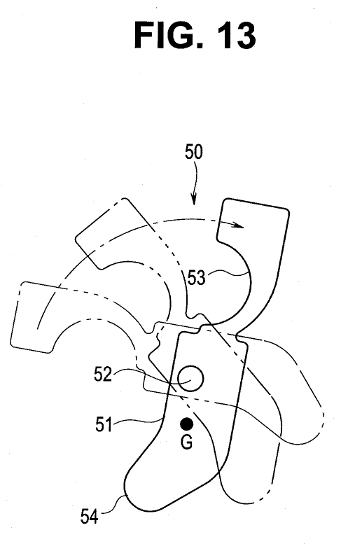

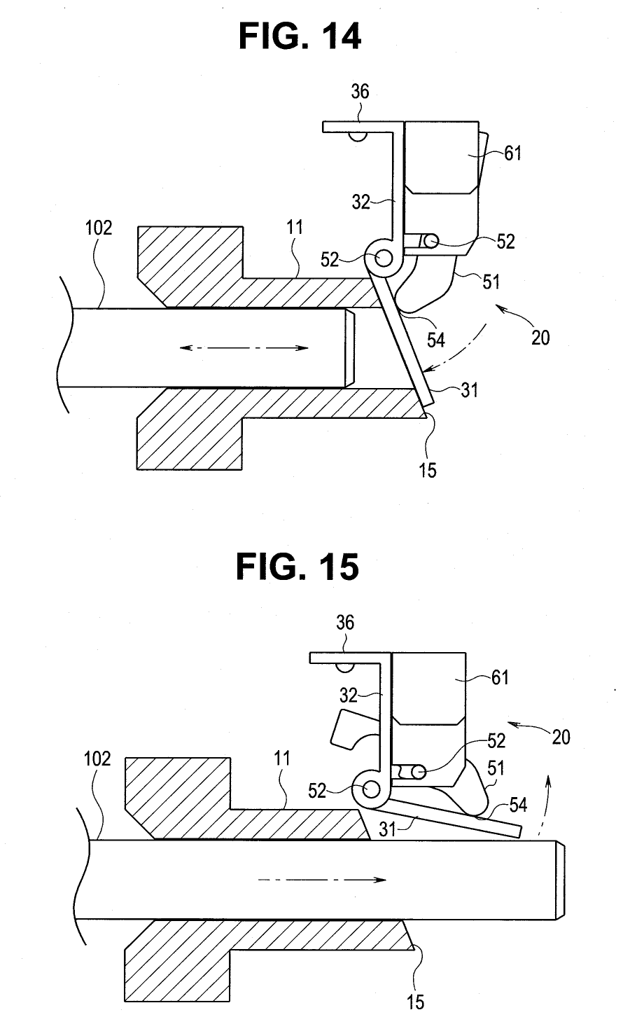

FIG. 13 is a side view illustrating a configuration of a turning member in a first modification, FIG. 14 is a cross-sectional view in an initial state where a light source connector of a connector of an endoscope is inserted into a light source connector receiving section in the first modification, and FIG. 15 is a cross-sectional view in an in-use state where the light source connector of the connector of the endoscope is inserted into the light source connector receiving section in the first modification.

As illustrated in FIG. 13, a turning member 50 may have a posture in which an abutment end portion 54 in a main body section 51 is positioned on a lower side under its own weight while remaining assembled to a support base 60 by providing the abutment end portion 54 below a turning shaft 52, contrary to the turning member 50 in the above-described embodiment, and having a center of gravity G set vertically below the turning shaft 52.

When the turning member 50 is thus configured, a light blocking unit 20 can be arranged on an upper side in a connector section 10, contrary to the light blocking unit 20 in the above-described embodiment, as illustrated in FIG. 14.

In an initial state where a light source connector 102 in a connector 101 of an endoscope 100 does not contact a light blocking plate 31 in a light blocking plate unit 30 at the time of insertion or removal when the light source connector 102 is inserted into or removed from a light source connector receiving section 11 (including the time of not-in-use), a stress to be turned in a direction in which the abutment end portion 54 in the main body section 51 abuts on and pushes down the light blocking plate 31 under an own weight of the turning member 50 is generated in addition to an urging force of a torsion spring 34 and a stress under an own weight of the light blocking plate 31.

As illustrated in FIG. 15, in an in-use state where the light source connector 102 is inserted into the light source connector receiving section 11 so that the connector 101 of the endoscope 100 is connected to the connector section 10 in the light source apparatus for endoscope 1, there occurs a state where the light blocking plate 31 is urged and turned to be raised upward against the urging force of the torsion spring 34, the stress under the own weight of the light blocking plate 31, and the stress to be turned under the own weight of the turning member 50.

Thus, an installation position of the light blocking unit 20 can be changed to an upper side (in the present modification) and a lower side (in the above-described embodiment) in the connector section 10 depending on a position of the center of gravity G with respect to the turning shaft 52 in the turning member 50.

As a result, the light source apparatus for endoscope 1 can selectively determine the installation position of the light blocking unit 20 on the upper side or the lower side in the connector section 10 to match a layout space restricted by an internal object.

### (Second Modification)

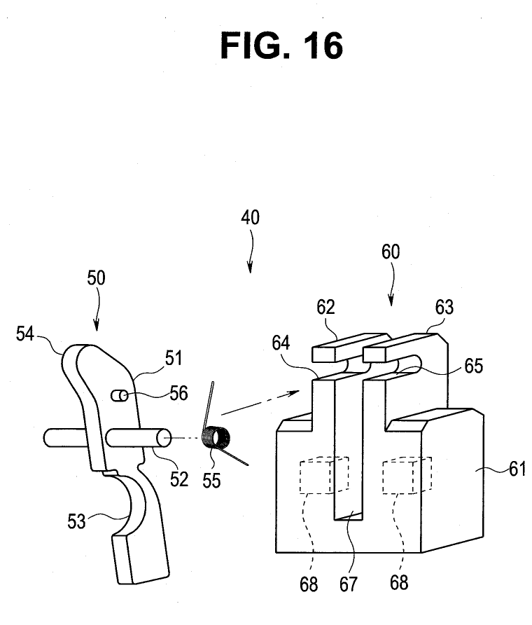

FIG. 16 is an exploded perspective view illustrating a configuration of an assist unit in a second modification.

As illustrated in FIG. 16, in an assist unit 40, a turning shaft 52 may be provided with a torsion spring 55 as an urging member configured to urge the turning member 50 in a direction in which an abutment end portion 54 in a main body section 51 abuts on the light blocking plate 31. Note that the main body section 51 is provided with a protrusion 56 configured to lock one end of the torsion spring 55, and a support base 60 is also provided with a protrusion not illustrated configured to lock the other end of the torsion spring 55.

As a result, in the assist unit 40, a stress to be turned by the abutment end portion 54 in the main body section 51 abutting on the light blocking plate 31 is increased so that the light blocking plate 31 can more reliably close an opening portion of a light source connector receiving section 11 to block light.

### (Third Modification)

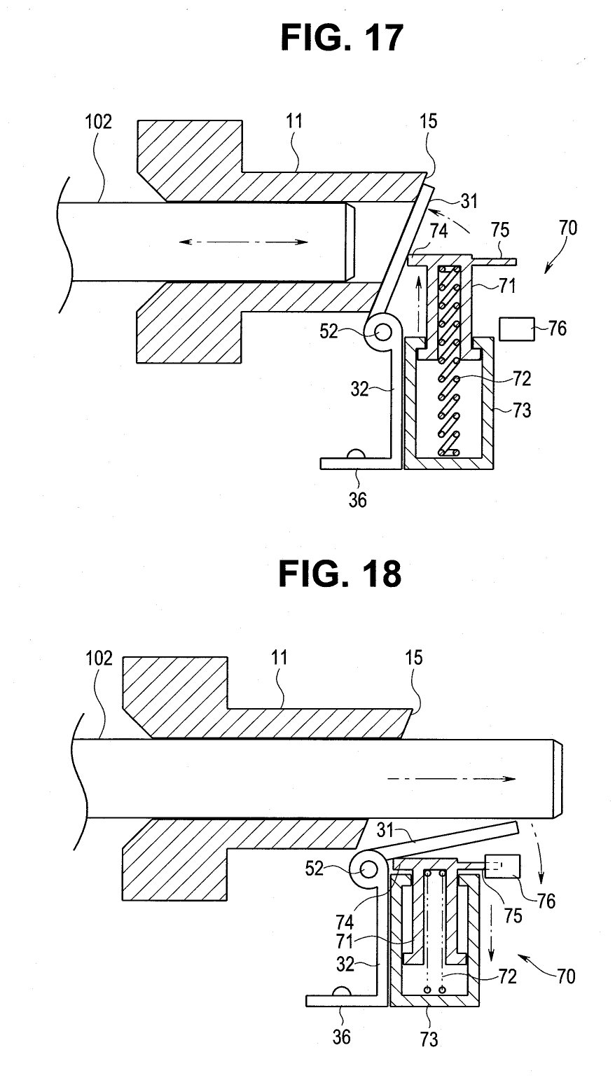

FIG. 17 is a cross-sectional view in an initial state where a light source connector of a connector of an endoscope is inserted into a light source connector receiving section in a third modification, and FIG. 18 is a cross-sectional view in an in-use state where the light source connector of the connector of the endoscope is inserted into the light source connector receiving section in the third modification.

As illustrated in FIGS. 17 and 18, an assist unit 70 includes a compression coil spring 72 as an urging member configured to urge a vertical direct-acting member 71 as a movable member, and the vertical direct-acting member 71 abuts on a light blocking plate 31 to close an opening portion of a light source connector receiving section 11 in addition to an urging force of a torsion spring 34 in a light blocking plate unit 30.

More specifically, the assist unit 70 includes a case body 73 as a drop preventing member configured to linearly guide the vertical direct-acting member 71 and prevent the vertical direct-acting member 71 from dropping out.

The vertical direct-acting member 71 is urged in such a direction as to protrude from the case body 73 by the compression coil spring 72, and includes an abutment section 74 configured to abut on the light blocking plate 31 and a protrusion 75 provided to extend in a horizontal direction from the opposite side to the abutment section 74. A photosensor 76 as a detection section configured to detect the protrusion 75 is provided.

The assist unit 70 enters a state where the light blocking plate 31 abutting on the abutment section 74 is pushed up not to be turned downward so that the opening portion of the light source connector receiving section 11 is closed with the vertical direct-acting member 71 protruding from the case body 73 by the urging force of the compression coil spring 72, illustrated in FIG. 17. At this time, the protrusion 75 is at a position where the photosensor 76 enters an open state, and it is detected that the connector 101 of the endoscope 100 has not been connected to the connector section 10.

In the assist unit 70, in an in-use state where a light source connector 102 is inserted into a light source connector receiving section 11 so that a connector 101 of an endoscope 100 is connected to a connector section 10 in a light source apparatus for endoscope 1, illustrated in FIG. 18, the vertical direct-acting member 71 is housed in the case body 73 and the light blocking plate 31 is turned downward against the urging force of the compression coil spring 72. At this time, when the protrusion 75 moves to bring the photosensor 76 into a close state, it is detected that the connector 101 of the endoscope 100 has been connected to the connector section 10.

Thus, the assist unit 70 is configured such that the vertical direct-acting member 71 is urged by the compression coil spring 72 in addition to the urging force of the torsion spring 34 in the light blocking plate unit 30 so that the light blocking plate 31 closes the opening portion of the light source connector receiving section 11 to block light.

Even if thus configured, the light source apparatus for endoscope 1 is configured such that a light blocking unit 20 configured to urge the light blocking plate 31 by the assist unit 70 in addition to the urging force of the torsion spring 34 in the light blocking plate unit 30 can reliably block illumination light from a light source at the time of lighting such that the illumination light does not leak when the connector 101 of the endoscope 100 is inserted or removed, including the time of not-in-use.

Although a configuration in which the assist unit 70 is provided on a lower side in the connector section 10 is illustrated, a configuration in which the assist unit 70 is provided on an upper side in the connector section 10 may be used, like in the first modification.

### (Fourth Modification)

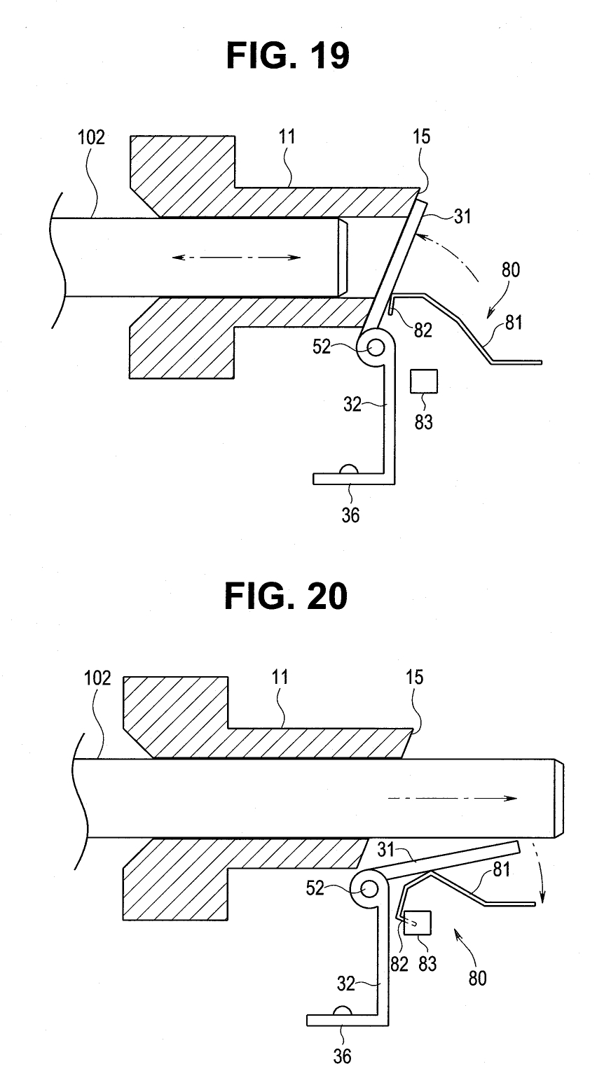

FIG. 19 is a cross-sectional view in an initial state where a light source connector of a connector of an endoscope is inserted into a light source connector receiving section in a fourth modification, and FIG. 20 is a cross-sectional view in an in-use state where the light source connector of the connector of the endoscope is inserted into the light source connector receiving section in the fourth modification.

As illustrated in FIGS. 19 and 20, an assist unit 80 includes a plate spring 81 as an urging member folded at a plurality of positions as a movable member, and the plate spring 81 abuts on a light blocking plate 31 to close an opening portion of a light source connector receiving section 11 in addition to an urging force of a torsion spring 34 in a light blocking plate unit 30. Note that a photosensor 83 as a detection section configured to detect an end portion 82 of the plate spring 81 is provided.

The assist unit 80 is brought into a state where the light blocking plate 31 is pushed up not to be turned downward by the plate spring 81 abutting on the light blocking plate 31 by an urging force of the plate spring 81 so that the opening portion of the light source connector receiving section 11 is closed, illustrated in FIG. 19. At this time, the end portion 82 of the plate spring 81 is at a position where the photosensor 83 enters an open state, and it is detected that a connector 101 of an endoscope 100 has not been connected to a connector section 10.

In the assist unit 80, in an in-use state where a light source connector 102 is inserted into the light source connector receiving section 11 so that the connector 101 of the endoscope 100 is connected to the connector section 10 in a light source apparatus for endoscope 1, illustrated in FIG. 20, the light blocking plate 31 is turned downward against the urging force of the plate spring 81. At this time, when the end portion 82 of the plate spring 81 moves to bring the photosensor 83 into a close state, it is detected that the connector 101 of the endoscope 100 has been connected to the connector section 10.

Thus, the assist unit 80 is configured such that the light blocking plate 31 closes the opening portion of the light source connector receiving section 11 to block light by the plate spring 81 in addition to the urging force of the torsion spring 34 in the light blocking plate unit 30.

Even if thus configured, the light source apparatus for endoscope 1 is configured such that the light blocking unit 20 configured to urge the light blocking plate 31 by the assist unit 80 in addition to the urging force of the torsion spring 34 in the light blocking plate unit 30 to close the opening portion of the light source connector receiving section 11 can reliably block illumination light from a light source at the time of lighting such that the illumination light does not leak when the connector 101 of the endoscope 100 is inserted or removed, including the time of not-in-use.

Although a configuration in which the assist unit 80 is provided on a lower side in the connector section 10 is illustrated, a configuration in which the assist unit 80 is provided on an upper side in the connector section 10 may be used, like in the first modification.

The invention described in the above-described embodiment is not limited to the forms, but various modifications can be made without departing from the scope and spirit of the invention in the implementation stage. Further, each of the above-described forms includes inventions in various stages, and various inventions can be extracted by an appropriate combination in a plurality of constitutional requirements to be disclosed.

For example, even if some constitutional requirements are deleted from all constitutional requirements illustrated in each of the forms, when described problems can be solved, and described effects are obtained, a configuration in which the constitutional requirements are deleted can be extracted as the invention.

According to the present invention, a light source apparatus for endoscope configured to more reliably block illumination light from a light source at the time of lighting such that the illumination light does not leak when a connector of an endoscope is inserted or removed, including the time of not-in-use, can be implemented.

What is claimed is:

1. A light source apparatus for endoscope comprising:
a light source configured to generate illumination light;
a light source connector receiving member into or from which a light source connector provided in a connector of an endoscope is inserted or removed;
a light blocking plate configured to be movable between a position where an opening portion of the light source connector receiving member is closed and a position where the opening portion is exposed and including an abutment surface configured to abut on the light source connector; and
a movable member configured to abut on a rear surface of the abutment surface of the light blocking plate, press the light blocking plate at the position where the opening portion is closed from a side of the rear surface in a closing direction in which the opening portion is closed with the light source connector not inserted into the light source connector receiving member, and make the light blocking plate movable to the position where the opening portion is exposed against the pressing in the closing direction when the light source connector inserted into the light source connector receiving member presses the light blocking plate by abutting on the abutment surface of the light blocking plate.

2. The light source apparatus for endoscope according to claim 1, further comprising
a first urging member configured to urge the light blocking plate in the closing direction in which the opening portion of the light source connector receiving member is closed.

3. The light source apparatus for endoscope according to claim 1, wherein
the movable member is provided to be turnable, and has a center of gravity set vertically below a turning shaft, to press the light blocking plate in the closing direction in which the opening portion of the light source connector receiving section is closed under an own weight of the movable member.

4. The light source apparatus for endoscope according to claim 1, further comprising
a second urging member configured to urge the movable member in a direction in which the light blocking plate is pressed in the closing direction in which the opening portion of the light source connector receiving member is closed.

5. The light source apparatus for endoscope according to claim 1, further comprising
a detection member configured to detect movement of the movable member corresponding to the insertion or removal of the light source connector into or from the light source connector receiving member.

6. The light source apparatus for endoscope according to claim 5, further comprising
a control section configured to control ON/OFF of the light source based on a detection result of the detection member.
